(19) United States
(12) Patent Application Publication
(10) Pub. No.: US 2014/0024036 A1
(43) Pub. Date: Jan. 23, 2014

(54) METHODS AND MATERIALS FOR QUANTIFICATION OF FUSOBACTERIUM NUCLEATUM DNA IN STOOL TO DIAGNOSE COLORECTAL NEOPLASM

(71) Applicant: GLC Biotechnology, Inc., Solon, OH (US)

(72) Inventors: NAIZHEN WANG, TWINSBURG, OH (US); BAOCHUAN GUO, SOLON, OH (US); YIDING LIU, TWINSBURG, OH (US)

(21) Appl. No.: 13/947,044

(22) Filed: Jul. 20, 2013

Related U.S. Application Data
(60) Provisional application No. 61/674,385, filed on Jul. 22, 2012.

(51) Int. Cl. C12Q 1/68

(52) U.S. Cl. CPC C12Q 1/6886
USPC 435/6.12

(57) ABSTRACT

Methods and materials for detecting colorectal cancer using DNA markers from human stool. More specifically, methods and materials for quantification of FNN bacteria DNA per unit stool weight as a molecular marker for CRC diagnosis.

### CROSS-REFERENCE TO RELATED APPLICATION

The present application claims priority to and the benefit of U.S. provisional patent application No. 61/674,385 filed Jul. 22, 2012, the entire disclosure of which is incorporated herein by reference.

### FIELD OF THE INVENTION

This invention provides methods for detecting the presence of colorectal neoplasm in patients by quantifying fusobacterium nucleatum DNA (FNN DNA) and other biomarkers such as human DNA markers and blood markers in the stool collected from the patients.

### BACKGROUND

Colorectal cancer (CRC) is the second deadliest cancer in USA. The good news is that the early detection along with resection is associated with a five-year survival rate of near 100%. All American over 50 are recommended to undergo CRC screening. Fecal occult blood tests (FOBTs) have been used to screen CRC for many years and continue to be one of the most frequently used screening tools. However, its screening accuracy is marginal. Due to limitations of FOBTs, colonoscopy is used as an alternative approach for CRC screening, but has the compliance disincentives including unpleasant cathartic preparation, invasive instrumentation, and small risk of harm. The limitations of existing options result in an unacceptable low patient compliance rate (only about 50% of Americans over 50 years currently undergo screening within the recommended intervals).There is a need to develop new screening methods that are more sensitive and specific, but non-invasive.

A number of molecular screening options have been proposed, among which fecal DNA testing is emerging as an attractive non-invasive procedure. First, stool screening is uniquely noninvasive, requires no unpleasant cathartic preparation, and can be performed on mailed-in specimens even without a physician office visit. Second, the use of DNA markers can make stool-based screening more attractive by greatly improving its sensitivity and specificity. An unmatched advantage of fecal DNA testing over other molecular methods such as blood testing is that fecal DNA testing can detect advanced adenoma (pre-cancer). Thus, fecal DNA testing can potentially be a preventive method. Recently, the US Multi-Society Task Force endorsed fecal DNA testing as an approach to screen CRC.

Fecal DNA testing is based on detecting tumor-associated DNA in stool. A typical fecal DNA assay consists of four steps. The first step is to collect stool from a patient. The second step is to transport the stool to a clinical Lab. The third step is to extract raw DNA from stool, followed by purification. The final step is to detect the markers (DNA analysis) from the stool DNA to identify “CRC positive” patients.

Gram-negative bacterial species fusobacterium nucleatumn (FNN) is an invasive, adherent, and pro-inflammatory anaerobic bacterium. It is common in dental plaque and there is a well-established association between FNN and periodontities. Kostic et al. and Castellarin et al. found that colorectal cancer tissues often contain FNN, while normal tissues do not. Based on the tissue study, it was speculated in a patent application that a positive detection of FNN in stool would indicate the presence of CRC.

Recently, we carried out a study to test this speculation with the stools collected from the individuals without CRC by detecting FNN DNA. We found that FNN DNA was positively detected in over 70% of the stools collected from the individuals without CRC and that a simple positive detection of FNN in stool cannot be used to detect CRC, as it leads to too many false positives.

The present invention provides novel methods for detecting CRC through fecal DNA testing. In a preferred embodiment, human stool FNN DNA quantification was deployed as an effective biomarker for colorectal neoplasm detection. And in another preferred embodiment, combining the quantitative FNN DNA marker with other biomarkers can detect CRC in a sensitive and specific manner

### SUMMARY OF THE INVENTION

The present invention provides methods for diagnosis of colorectal neoplasm in subjects, preferably human.

In one aspect, the present invention provides methods for diagnosing colorectal neoplasm in a subject. In some embodiments, the methods involve obtaining stool sample from a subject, and quantifying fusobacterium nucleatum (FNN) DNA present in the stool sample, where the amount of fusobacterium nucleatum (FNN) DNA detected in a given amount of stool indicates the diagnosis of colorectal neoplasm.

In some embodiments, quantification of fusobacterium nucleatum (FNN) DNA present in given amount of stool sample in combination with the presence of one or more other molecular biomarkers is indicative of colorectal neoplasm presenting in the subject.

In some embodiments, the one or more other molecular biomarkers that may be used in combination with quantification of fusobacterium nucleatum (FNN) DNA present in given amount of stool sample include, but not limited to, human DNA mutations, quantity of human DNA in a given amount of stool sample, quantity of total DNA in a given amount of stool sample, fecal occult blood markers, and human genes having aberrant methylation.

The methods are not limited to particular human DNA mutation markers. In some embodiments, human DNA mutation markers that may be used in combination with quantification of FNN DNA present in given amount of stool sample include, but not limited to, K-ras, APC, melanoma antigen gene, P53, BRAF, BAT26 and PIK3CA. In some embodiments, human DNA mutation marker used in combination with the quantity of FNN DNA present in a given amount of stool sample is K-ras.

In some embodiments, quantification of fusobacterium nucleatum (FNN) DNA present in a given amount of stool sample could be performed using methods that are well known to the person skilled in the art, including, but not limited to, quantitative real time polymerase chain reaction (qPCR).

In some embodiments, quantity of human DNA in a given amount of stool sample could be obtained using methods that are well known to the person skilled in the art, including, but not limited to, quantitative real time polymerase chain reaction (qPCR).

In some embodiments, quantity of total DNA in a given amount of stool sample could be obtained using methods that are well known to the person skilled in the art, including, but not limited to, Ultra-violet (UV) spectrometry.

The methods are not limited to particular fecal occult blood markers. In some embodiments, fecal occult blood markers that may be used in combination with quantification of fusobacterium nucleatum (FNN) DNA present in a given amount of stool sample include, but not limited to, hemoglobin, alpha-defensin, calprotectin, alpha1-antitrypsin, albumin, MCM2, transferrin, lactoferrin, and lysozyme.

The methods are not limited to particular aberrant methylated human gene markers. In some embodiments, human genes having aberrant methylation that may be used in combination with quantification of fusobacterium nucleatum (FNN) DNA present in given amount of stool sample include, but not limited to, BMP-3, BMP-4, SFRP2, vimentin, septin9, ALX4, EYA4, TFPI2, NDRG4, HLTF, and FOXE1.

The methods are not limited to the marker combinations above. Other colorectal neoplasm specific markers could be combined with quantification of fusobacterium nucleatum DNA in a given amount of stool sample to further improve the diagnosis.

In some embodiments, the colorectal neoplasm is premalignant including adenomatous lesion and polyp. In some embodiments, the colorectal neoplasm is malignant.

When using the quantity of fusobacterium nucleatum (FNN) DNA present in given amount of stool sample as an indicative biomarker for colorectal neoplasm detection, the quantity of FNN DNA present in a given amount of stool must be above a certain level (threshold) to distinguish subjects with colorectal neoplasm from those without colorectal neoplasm.

In some embodiments, said level (threshold) was experimentally determined based on the sample sets tested. However, the present invention will not be limited to a specific threshold. And this threshold is subject to experimental conditions and the methods used to distinguish subjects with colorectal neoplasm from those without colorectal neoplasm.

In some embodiments, after collecting stool sample from the subject, the stool sample is incubated in a preservation buffer to protect DNA from degradation during transportation and storage. The preserved stool DNA is then extracted and purified using protocols that is well known to the person skilled in the art, including, but not limited to, iso-propanol/ethanol precipitation, QIAgen QIAamp DNA stool kit, Zymo Research DNA clean and concentrator kit, magnetic beads based automated DNA collection and purification system, and sequence-specific capture methods. In some embodiments, stool DNA samples are further treated and purified to remove PCR inhibitors using, for example, phenol-chloroform extraction, and/or filtered with Zymo HRC column.

In some embodiments, when a colorectal neoplasm is identified with the methods provided by the present invention, additional clinical techniques are performed to characterize the colorectal neoplasm.

In certain embodiments, the present invention provides methods for monitoring the treatment of colorectal cancer. For example, in some embodiments, the methods may be performed before, during and/or after a treatment to monitor treatment success. In some embodiments, the methods are performed at intervals on disease free patients to insure or monitor treatment success.

### BRIEF DESCRIPTION OF THE DRAWINGS

FIG. 1 represents the detection of FNN DNA in stools of 38 patients without CRC. FNN(+) and FNN (−) indicate the positive or negative detection of FNN DNA in stool, respectively. 32 μg and 800 μg indicate the amount of equivalent stool DNA input into PCR.

FIG. 2 represents the relative FNN DNA abundance comparison in human fecal samples from CRC (colorectal cancer) and normal subjects. The data were plotted by Graphpad Prism® 5 software with logarithm Y axis.

TABLE 1 represents the results of detecting colorectal neoplasm using quantitative detection of FNN DNA in a given amount of stool alone and in combination with other stool DNA markers. FNN 112 stands for the quantity of the FNN DNA fragment of 112bp in 32 μg stool. ACTB is representing the quantity of human DNA (human β-actin gene or ACTB) detected in 32 μg stool. K-ras is referred to as any mutation in codon 12 or codon 13 of the human oncogene K-ras detected in 1.6 mg stool. Total DNA is the quantity of total stool DNA in 32 μg stool.

### Definitions

Neoplasm—Neoplasm is an abnormal mass of tissue as a result of neoplasia. Neoplasia is the abnormal proliferation of cells. Prior to neoplasia, cells often undergo an abnormal pattern of growth, such as metaplasia or dysplasia. However, metaplasia or dysplasia do not always progress to neoplasia. The growth of neoplastic cells exceeds and is not coordinated with that of the normal tissues around it. The growth persists in the same excessive manner even after cessation of the stimuli. It usually causes a lump or tumor. Neoplasms may be benign, premalignant (carcinoma in situ) or malignant (cancer).

CRC—Colorectal cancer, commonly known as colon cancer or bowel cancer, is a cancer from uncontrolled cell growth in the colon or rectum (parts of the large intestine), or in the appendix.

FOBT—Fecal occult blood (FOB) refers to blood in the feces that is not visibly apparent. A fecal occult blood test (FOBT) checks for hidden (occult) blood in the stool (feces). Newer tests look for globin, DNA, or other blood factors including transferrin, while conventional stool guaiac tests look for heme.

Gram-negative bacteria—Gram-negative bacteria are bacteria that do not retain crystal violet dye in the Gram staining protocol. The test itself is useful in classifying two distinct types of bacteria based on the structural differences of their bacterial cell walls. The pathogenic capability of Gram-negative bacteria is often associated with certain components of Gram-negative cell envelope, in particular, the lipopolysaccharide layer (also known as LPS). In humans, LPS triggers an innate immune response characterized by cytokine production and immune system activation. Inflammation is a common result of cytokine production, which can also produce host toxicity.

Anaerobic Bacteria—Anaerobic bacteria are bacteria that do not require oxygen for survival. Anaerobic bacteria cannot bear oxygen and may die if kept in an oxygenated environment.

Gram-negative anaerobic bacilli may cause infections anywhere in the body; the most common types are oral and dental, pleuropulmonary, intra-abdominal, female genital tract and skin, soft tissue and bone infections. They may play a role in such diverse pathologic processes as periodontal disease and colon cancer.

Invasive bacteria—Invasive bacteria are pathogens that can invade parts of the body where bacteria are not normally present, such as the bloodstream, soft tissues like muscle or fat, and the meninges (the tissues covering the brain and spinal cord).

Fusobacterium nucleatum (FNN)—Fusobacterium is a genus of anaerobic, Gram-negative bacteria with invasive and adherent characteristics. Strains of fusobacterium contribute to several human diseases, including periodontal diseases, Lemierre's syndrome, and topical skin ulcers. Fusobacterium should always be treated as a pathogen. In 2012, researchers discovered that fusobacterium nucleatum (FNN, a strain of fusobacterium) flourishes in colon cancer cells, and is often also associated with ulcerative colitis.

PCR—The polymerase chain reaction (PCR) is a biochemical technology in molecular biology to amplify a single or a few copies of a piece of DNA across several orders of magnitude, generating thousands to millions of copies of a particular DNA sequence.

qPCR—In molecular biology, real-time polymerase chain reaction, also called quantitative real time polymerase chain reaction (qPCR) is a laboratory technique based on the PCR, which is used to amplify and simultaneously quantify a targeted DNA molecule. The procedure follows the general principle of polymerase chain reaction; its key feature is that the amplified DNA is detected as the reaction progresses in real time. This is a new approach compared to standard PCR, where the product of the reaction is detected at its end.

For one or more specific sequences in a DNA sample, qPCR enables both detection and quantification. The quantity can be either an absolute number of copies or a relative amount when normalized to DNA input or additional normalizing genes.

Ct Number and Quantification using qPCR—qPCR can be used to quantify DNA by two methods: relative quantification and absolute quantification. Relative quantification is based on internal reference genes to determine fold-differences of the target gene. Absolute quantification gives the exact number of target DNA molecules by comparison with DNA standards.

During the exponential amplification phase, the sequence of the DNA target doubles every cycle. So the general principle of DNA quantification by real-time PCR relies on plotting fluorescence against the number of cycles on a logarithmic scale (with a base of 2).

A threshold for detection of DNA-based fluorescence is set slightly above background. The number of cycles at which the fluorescence exceeds the threshold is called the cycle threshold (Ct). Smaller Ct value represents more starting target DNA template (less amplification needed to exceed the threshold). For example, a DNA sample A whose Ct precedes that of another sample B by 3 cycles contained 2³=8 times more template. In another words, if we assign sample A as reference (the amount of DNA template in sample A as 1), the difference in Ct (ACt) equals CtB−CtA=3, the amount of starting DNA template in sample B relative to sample A could be calculated as 2^{-ΔCt}=2⁻³=0.125.

However, the efficiency of amplification is often variable among primers and templates. Therefore, the efficiency of a primer-template combination is assessed in a titration experiment with serial dilutions of DNA template to create a standard curve of the change in Ct with each dilution. The slope of the linear regression is then used to determine the efficiency of amplification, which is 100% if a dilution of 1:2 results in a Ct difference of 1.

Oncogene and K-ras mutation—An oncogene is a gene that has the potential to cause cancer. In tumor cells, they are often mutated or expressed at high levels (producing higher than normal levels of corresponding protein products).

A proto-oncogene is a normal gene that can become an oncogene due to mutations or increased expression. The proto-oncogene can become an oncogene by a relatively small modification of its original function (such as a single nucleotide substitution, which in turn causing a single amino acid substitution in protein product). A mutation within a proto-oncogene can cause a change in the protein, causing an increase in protein (enzyme) activity and/or a loss of regulation.

GTPase KRas also known as V-Ki-ras2 Kirsten rat sarcoma viral oncogene homolog and KRAS, is a protein that in humans encoded by the K-ras gene. The protein product of the normal K-ras gene acts as a molecular on/off switch in normal tissue signaling. The mutation of K-ras gene is an essential step in the development of many cancers including lung adenocarcinoma and colorectal carcinoma.

### DETAILED DESCRIPTION OF THE INVENTION

### Fecal DNA Testing

An effective way to detect colorectal cancer (CRC) at early stages is population screening. Colonoscopy and fecal occult blood testing (FOBT) are commonly used tools for CRC screening, but the adherent rates of both approaches are low due to the invasiveness and expense of colonoscopy and the low sensitivity of FOBT.

Fecal DNA testing is emerging as an attractive non-invasive procedure for CRC screening because of its high sensitivity, noninvasive and low cost characteristics. An unmatched advantage of fecal DNA testing over other molecular methods such as blood testing is that fecal DNA testing can detect advanced adenoma (pre-cancer) as described in the present invention. Thus the detection of advanced adenoma by fecal DNA testing along with resection could potentially be a preventive method for controlling CRC.

Fecal DNA testing is based on detecting tumor-originated or tumor-associated DNA in stool. A typical fecal DNA assay consists of four steps. The first step is to collect stool from a patient. The second step is to transport the stool (with proper DNA preserving buffer in a collection tube) to a clinical Lab. The third step is to extract DNA from stool, followed by purification and concentration. The final step is to detect the tumor specific markers (DNA analysis) from the stool DNA to identify “CRC positive” patients.

### Human DNA Markers for CRC Screening

Human DNA alterations have been the choice of biomarkers for CRC screening. There are two major types of DNA alterations. The first is mutations. Genomic mutations driving adenomas to carcinomas have been proposed for CRC and thus can be used as the markers of CRC screening. K-ras, p53, BAT-26, and APC are frequently mutated in CRC, melanoma antigen gene, BRAF, and PIK3CA were also reported as CRC specific mutation markers.

Early studies focused on K-ras, as its mutations occur in about 40% of CRC, over 90% of which are confined to its codons 12 and 13. Mutations in p53 occur in 50-60% of CRC. 10-15% of sporadic CRC are microsatellite instability high (MSI-H), including 3-6% of hereditary nonpolyposis colorectal cancer (HNPCC). Studies suggest that BAT-26 alterations were present in 95% of MSI-H CRC, but not present in normal tissues, making it a good marker of MSI-H. APC mutations indicate colorectal tumors at the earliest stage. Mutations in APC initiate 80-85% of colorectal neoplasia, making them the most sensitive CRC biomarker. Studies had shown that no APC mutations were present in the samples from patients without neoplasia, suggesting that they are tumor-specific.

Early versions of fecal DNA testing used DNA mutations as markers for CRC screening. However, studies had shown that this panel of mutation markers was not sufficiently sensitive to detect CRC. It was found that fecal DNA testing with the mutation markers only detected 52% of invasive cancer and 18% of advanced adenoma. One reason for this is that only a small number of mutations in p53 and APC were actually included in the test, while hundreds of other APC and p53 mutations were not included. To improve the detection sensitivity, one must utilize a much larger number of mutations as markers, but this is currently not feasible, for it is challenging and expensive to simultaneously detect hundreds of mutations with current technologies.

The second type of DNA alteration is aberrant methylation. Methylation in a number of genes is frequently associated with CRC. In a seminar report, Muller et al. reported the use of one sFRP2 gene for CRC screening. Later, Lenhard et al. reported the use of the HIC1 gene for CRC screening. Thereafter, many studies involving the use of methylated DNA as CRC screening markers were reported.

Although methylation markers are more sensitive comparing to mutation markers, their specificity is poorer, especially when they are used as the stool marker for CRC screening. For instance, Muller and coworkers reported that methylation in SFERP2 occurred in the stool DNA of 77% of the CRC patients, but also in the stool DNA of 23% of the patients without tumor.

One reason for this specificity problem is that aging can also lead to DNA methylation in normal tissues. In addition, a methylated gene may be colon-cancer specific, but may not be tumor-specific in other organs (in other words, this gene can be methylated in normal tissues of other organs). Methylated DNA originated from other organs can get into stool as well. Another challenge in methylation analysis is that bisulfite treatment could destroy more than 90% of DNA (Note that most methylation analysis methods are based on bisulfite treatment to convert C to U, while the methylated C resists such changes). This problem becomes magnified in fecal DNA testing, as stool samples often contain little human DNA. In other words, after bisulfite treatment of DNA, there may not be even one copy of methylated DNA left for analysis.

Another human DNA marker is the quantity of human DNA in stool. It was found that the quantity of human DNA in the stool of the CRC patients was more abundant than that extracted from the stool of controls without CRC. However, the quantity of human DNA in stool is not a sensitive biomarker by itself and can only be used with other markers. Clearly, there is a great need for new stool DNA markers for CRC screening.

### Fecal Fusobacterial DNA as a Novel DNA Marker for CRC Screening

The present invention provides methods for fecal DNA test based CRC screening. In a preferred embodiment, a novel quantitative fusobacterial DNA marker is used to detect colorectal neoplasm.

Fusobacterium is a genus of gram-negative, filamentous, anaerobic bacteria found as normal flora in the mouth and large bowel, and often in necrotic tissue. In recent studies, a comparison of microbial ribonucleic acids (RNA) between colorectal carcinoma (CRC) tissue and adjacent normal control tissue found the over-representation of fusobacterium nucleatum (FNN) in CRC tissue. And it was speculated that a positive (qualitative) detection of fusobacterium nucleatum (FNN) in stool would also indicate the presence of CRC. To validate this speculation, we carried out a study with the stools collected from individuals without CRC by detecting FNN DNA those stool samples. According to our results, FNN DNA was positively detected in about 70% of the stools collected from controls without CRC. This demonstrated that a simple positive (qualitative) detection of FNN DNA in stool can not be used to detect CRC, as it leads to too many false positives. One of the reasons that FNN DNA can also be present in the stools of people without CRC is that the stool DNA can be originated from other organs including mouth. It was reported that some of the DNA originated from other organs could survive and get into stool.

Since FNN DNA is present in the stool of most of the individuals without CRC, we have performed a systematic study to investigate the best way to utilize FNN DNA as a stool biomarker for CRC screening. In this study, we quantified FNN DNA in the stool collected from 21 patients with CRC and 38 individuals without CRC, respectively. We found that the best biomarker for this set of samples involving FNN DNA is FNN DNA quantity/per unit weight of stool, for this single marker alone could detect nearly 60% of CRC with better than 85% specificity (as shown in TABLE 1). In fact, the sensitivity and specificity are better than those of K-ras mutation markers (data not shown).

More importantly, we found that this DNA marker can serve as an anchor marker of a panel of the stool biomarkers for CRC screening. We discovered that a panel of markers with FNN DNA as an anchor could detect 95% of CRC with a detection specificity of better than 80%. Other markers in this panel include human DNA quantity/per unit weight of stool, the total quantity of stool DNA/per unit weight of stool. To the best of our knowledge, this may be the simplest panel of markers that yield the highest detection sensitivity with such a high specificity. In addition, since there are no methylation markers in this panel, the quantity of stool used to extract DNA can be reduced by 10-20 folds, greatly reducing the complexity and cost associated with the extraction/purification of DNA from stool and making automation of this process possible.

Therefore, the present invention, in some embodiments, provides a quantitative assay using fucobacterium nucleatum (FNN) DNA in a given amount of stool as a marker for detecting colorectal neoplasm. In some other embodiments, the present invention also provides an assay combining quantitative detection of fucobacterium nucleatum (FNN) DNA in a given amount of stool with other biomarkers including, but not limited to, quantification of total human DNA in stool, quantification of total DNA in stool, fecal occult blood markers, human DNA mutation markers, and aberrant methylated human gene markers, yielding an inexpensive solution for sensitive detection of colorectal neoplasm.

Comparing with the existing methods, the present invention does not increase the cost associated with retrieving and purifying DNA from stool. Yet, its benefits are enormous and this may finally provide a practical solution to overcome the last major barrier to fecal DNA testing.

### EXPERIMENTAL

### EXAMPLE 1

We first tested whether a positive detection (qualitative detection) of FNN in stool indicates the presence of CRC. FNN DNA originated from other organs can survive and get into stool. In other words, if FNN is present in other organ tissues (even if it is colon-tumor specific), the stool collected from those without CRC may contain FNN as well.

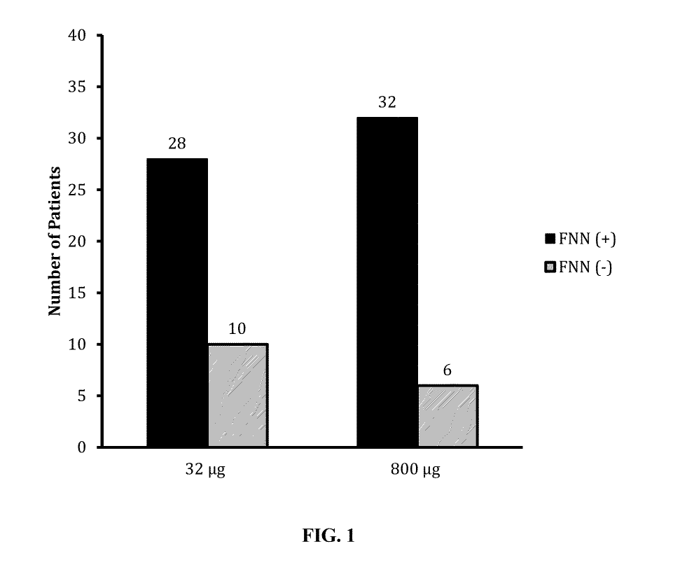

Stool samples collected from 38 patients without CRC were studied. FIG. 1 displays the number of positive detection of FNN DNA from the stools of the controls without CRC by qPCR. As shown in FIG. 1, FNN DNA was positively detected from 28 out of 38 stool samples (73%) when 32 μg equivalent stool DNA were input into qPCR, and 32 out of the 38 stool samples (84%) when 800 μg equivalent stool DNA was input into qPCR, respectively. This result confirmed our concerns that the stool collected from a large percentage of the subjects without CRC could contain FNN DNA as well, and that a positive detection (qualitative detection) of FNN DNA from stool alone cannot be used as a stool marker for CRC screening.

All stool samples were preserved in a preservation buffer right after collection and stored at −80° C. until use. After thawed and thoroughly homogenized, stool lysate (equivalent to 250 mg original stool) was transferred into a 15 mL centrifuge tube. Stool lysate was treated with 10 μg/mL RNase A (Qiagen) for 1 hour at room temperature and then 200 μg/mL proteinase K (Qiagen) for 2 hours at 56° C. A phenol-chloroform extraction was performed to remove impurities including proteins, fats, metabolic products and solid matters. Clarified stool lysate (equivalent to 100 mg stool) was then loaded onto a magnetic beads based automatic DNA extraction and purification system (Kingfisher Flex, Thermo Scientific) for further isolation. Extracted stool DNA was eluted in 500 uL Tris buffer and further purified using Zymo HRC column (Zymo Research) to remove PCR inhibitors. Purified stool DNA is ready for PCR.

We attempted to use the published PCR primers to amplify FNN DNA, but the published primers did not work well in our hands. Based on the genomic sequence of fucobacterium nucleatum (FNN), we designed our own primers to amplify FNN DNA from stool samples.

In this study, the genomic FNN DNA was subjected to qPCR to amplify a 112bp fragment of nusG gene (Genbank Accession Number AAL94126.1) of FNN (ATCC 25586), for DNA can be preserved after stool collection and thus is more stable than other molecular markers in stool. The primers used to amplify the 112-bp fragment were 5′-CAACCATTA CTTTAACTCTACCATGTTCA-3′ and 5′-ATTGACTTTACTGAGGGAGATTATGTAAAA-3′. qPCR was carried out using 32 μg or 800 μg stool equivalent DNA, 0.5 μM primer pairs, and 1× Precision Melt SuperMix (Bio-Rad) in a total volume of 20 μL. PCR cycling conditions for FNN amplification were 95° C. for 10 min followed by 60 cycles of 95° C. for 20 s, 57° C. for 30 s and 72° C. for 45 s, and ended by a melting curve from 60° C. to 95° C.

### EXAMPLE 2

Considering the result of Experiment 1 that a positive detection (qualitative detection) of FNN DNA from stool cannot distinguish the CRC patients from those without CRC, in this study, we used qPCR to quantify FNN DNA in a given amount of the stool samples collected from 21 patients with CRC (21 stool samples) and 38 controls without CRC. The 112bp FNN DNA fragment was amplified.

We normalized the quantity of FNN DNA against the unit weight of stool and discovered that this normalization strategy yielded the best results for CRC screening with the tested samples (as shown in TABLE 1).

We measured the relative abundance of FNN DNA among all stool samples and determined the cutoff threshold of the relative abundance that can distinguish CRC patients from those without CRC. Experimentally, for each stool sample, 32 μg stool equivalent of DNA were input into qPCR. The amount of DNA presented in stool were determined and represented in cycle threshold (Ct) numbers. We assigned the stool sample with the most abundant FNN DNA (with the smallest Ct) as a reference sample. The relative abundance of FNN DNA in other samples (aka “test sample”) was determined by measuring the difference (ACt) between the reference sample and a test sample. The relative abundance of FNN DNA in a test sample to FNN DNA in the reference sample was obtained by calculating 2^{-ΔCt}. Finally we multiplied all calculated relative abundances by 10,000 to make them all in whole numbers. The data were plotted by Graphpad Prism® 5 software with logarithm Y axis.

It should be noted that absolute quantification could be easily performed with a FNN DNA standard curve prepared in known FNN DNA concentrations. As a standard protocol known to those skilled in the art, the conversion of Ct number of each sample to absolute quantity of DNA is purely mathematical and the substitution of Ct and ΔCt to absolute DNA concentration will not change the calculated results and the following statistical analysis.

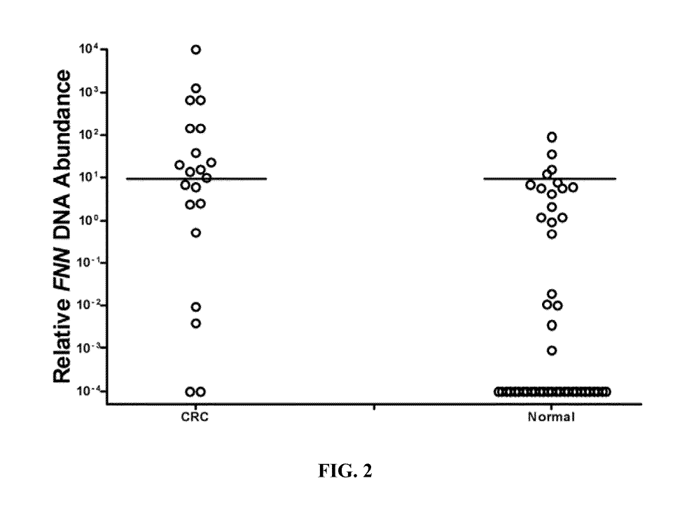

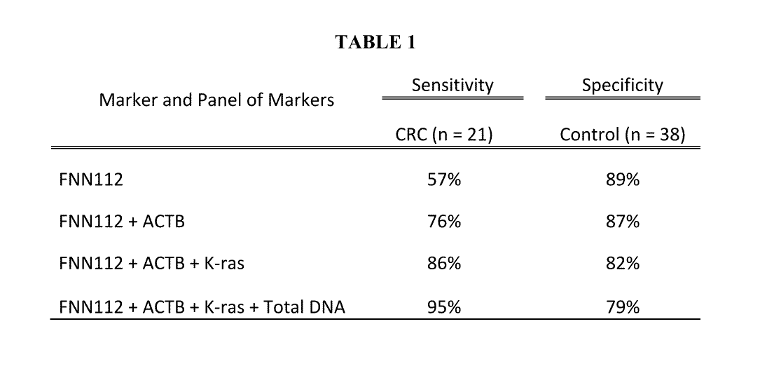

As shown in FIG. 2, the distribution of the measured FNN relative abundances in each group was non-parametric. By using Kruskal-Wallis test, we demonstrated that the difference of FNN abundance among three groups was significant (p<0.0001). Dunn's multiple comparison test further showed that a significant difference of FNN abundance exists between CRC patients and normal subjects (p<0.001). Moreover, we established a cutoff to distinguish CRC patients from those without CRC. Among 21 CRC patients, 12 of them have the relative abundance of FNN DNA/per stool weight above the cutoff; while among stool samples collected from 38 controls without CRC, only 4 have the values above the cutoff (FIG. 2). This corresponded to a sensitivity of 57% and specificity of 89.5% (TABLE 1).

All stool samples were collected and processed as mentioned in EXAMPLE 1. Stool DNA was extracted and purified as mentioned in EXAMPLE 1, too.

The genomic FNN DNA was subjected to qPCR to amplify 1 fragment of nusG gene (Genbank Accession Number AAL94126.1) of FNN (ATCC 25586). The primers used for amplification were 5′-CAACCATTACTT TAACTCTACCATGTTCA-3′ and 5′-ATTG ACTTTACTGAGGGAGATTATGTAAAA-3′. Quantitative PCR was carried out using 32 μg stool equivalent DNA, 0.5 μM primer pairs, and 1× Precision Melt SuperMix (Bio-Rad) in a total volume of 20 μL. PCR conditions for FNN amplification were 95° C. for 10 min followed by 60 cycles of 95° C. for 20 s, 57° C. for 30 s and 72° C. for 45 s, and ended by a melting curve from 60° C. to 95° C.

### EXAMPLE 3

No single marker can yield a perfect detection for CRC screening and a panel of markers may yield a better sensitivity. The quantity of FNN DNA/per unit stool weight is a specific marker with a sensitivity of better than 55%, making it an ideal anchor marker to be used with others to form a marker panel.

The markers studied along with FNN DNA were the quantity of human DNA/per unit stool weight, the quantity of total DNA/per unit stool weight, and k-ras mutations. We first combined quantity of FNN DNA with quantity of human DNA to form a duo-marker panel. As seen from TABLE 1, the sensitivity increased to 76% with this duo-marker panel from 57% when only quantification of FNN DNA was used as the marker, while the specificity slightly decreased to 87% from 89%. Then we added the k-ras mutations to the marker panel, leading to improvement of the detection sensitivity to 86% with a specificity of 82%. Finally, we added total stool DNA to the marker panel, the sensitivity increased to 95% with a specificity of 79%. To the best of our knowledge, this is the simplest panel of markers, which can achieve such a high sensitivity and specificity for CRC screening. Moreover, no methylated DNA markers were used. Clearly, a combination of quantification of FNN DNA with other stool markers could greatly enhance the detection sensitivity for CRC screening without significantly decreasing the screening specificity.

Please note that the “unit stool weight” used for normalizing Fecal FNN DNA, Fecal Human DNA and Total Fecal DNA could be of different value. As well known to the person skilled in the art, the primer efficiency in PCR amplification could vary significantly from gene to gene, as well as for each specific targeted sequence. As a result, the sensitivity of each qPCR is closely related to the target gene and specific sequence amplified. And the proper threshold and normalization unit should be experimentally determined by the person skilled in the art for each targeted markers.

The present invention should not be limited in the targeted sequences mentioned above. Any new genes and/or sequences of fusobacterium nucleatum DNA, and fecal human DNA, if being found to be compatible with the method of present invention, could be added in the panel. The threshold and unit for normalization should be adjusted accordingly by the person skilled in the art.

All stool samples were collected and processed as mentioned in EXAMPLE 1. Stool DNA was extracted and purified as mentioned in EXAMPLE 1, too.

The genomic FNN DNA was subjected to qPCR to amplify 1 fragment of nusG gene (Genbank Accession Number AAL94126.1) of FNN (ATCC 25586). The primers used for amplification were 5′-CAACCATTACTTTAACTCTACCATGTTCA-3′ and 5′-ATTG ACTTTACTGAGGGAGATTATGTAAAA-3′. Quantitative PCR was carried out using 32 μg stool equivalent DNA, 0.5 μM primer pairs, and 1× Precision Melt SuperMix (Bio-Rad) in a total volume of 20 μL. PCR conditions for FNN amplification were 95° C. for 10 min followed by 60 cycles of 95° C. for 20 s, 57° C. for 30 s and 72° C. for 45 s, and ended by a melting curve from 60° C. to 95° C.

The primers used for amplifying human β-actin gene ACTB were 5′-GGTAGGTTTGTA GCCTTCATCACG-3′ and 5′-CTTGAGAGGTAGAGTGTGGTGTTG-3′. qPCR was carried out using 32 μg stool equivalent DNA, 0.5 μM primer pairs, and 1× Hype-It-HRM (Qiagen) in a total volume of 20 μL. PCR cycling conditions for ACTB were 95° C. for 10 min followed by 50 cycles of 95° C. for 20 s, 62° C. for 30 s and 72° C. for 45 s, and ended by a melting curve reading from 60° C. to 95° C. A standard curve was prepared and amplified on the same plate using commercial human genomic DNA from Promega for absolute quantification.

The oncogene K-ras mutation in codon 12 or codon 13 was determined by qPCR amplification in conjunction with peptide nucleic acid (PNA) clamping of wild-type (WT) allele. The primers used for amplifying a 157-bp K-ras fragment were 5′-ATCGTCAAGGCACTCTTGCCTAC-3′ and 5′-GTACTGGTGGAGTATTTGATAGTG-3, and the PNA-Clamp (H₂N-TACGCCACCAGCTCC-CO₂H) was used to inhibit the annealing and amplification of the wide-type K-ras allele (thus improving the amplification and detection of the allele with mutations). qPCR was carried out in a total volume of 20 μL, containing 1.6 mg fecal equivalent DNA, 1× Type-It-HRM master mix (Qiagen), 0.25 μM of the each primer, in the presence or absence of 1.25 μm PNA (BioSynthesis). PCR cycling conditions for KRAS amplification were 95° C. for 10 min followed by 60 cycles of 95° C. for 20 s, 71° C. for 30 s and 60° C. for 60 s, and ended by a melting curve from 60° C. to 95° C.

The quantity of total stool DNA (including human and bacterial DNA, cellular DNA and free circulating fragments) was measured using Ultra-violet (UV) spectrometry.

### CITATION LIST

Patent Literature
US 2011/0236916 A1, ZOU et al., 2011
WO 2012/045150 A1, HOLT et al., 2012

Non Patent Literature
Robertson K D 2005 Nature Reviews Genetics; Vol. 6: 597-610
Sato F, Meltzer S J 2005 American Cancer Society; Vol. 106: 483-493
Levin B, et al. 2008 CA Cancer J Clin., Vol 58(3), 130-160
Ahlquist D A, et al. 2000 Gastroenterology, 119, 1219
Sidransky D, et al. 1992 Science, 256, 102
Dong S M, et al. 2001 J. Natl. Cancer Inst. 93, 858
Traverso G, et al. 2002 The Lancet, 359, 403
Traverso G, et al. 2002 N. Engl. J. Med. 346, 311
Ahlquist D A 2000 BMJ, 321, 254
Galistri D, et al. 2003 Clin. Gastroenterol Hapatol. 1, 377
Tagore K S, et al. 2003 Clin. Colorectal Cancer, 3, 47
Brand R E, et al. 2003 American Journal of Gastroenterology, 3, 1339
Whitney D, et al. 2004 Journal of Molecular Diagnostics, 6, 386
Imperiale T F, et al. 2004 N. Engl. J. Med. 351, 2704
Zou H, et al. 2009 Gastroenterology, Vol 136(2), 459-470
Muller H M, et al. 2004 Lancet, Vol 363, 1283-1285
Lenhard K, et al. 2005 Clin Gastroenterol Hepatol., Vol 3(2), 142-149
Chen W D, et al. 2005 J Natl Cancer Inst. Vol 97(15), 1124-1132
Zou H, 2007 Clin Chem., Vol 53(9), 1646-1651
Itzkowitz S, et al. 2008 Am J Gastroenterol., Vol 103(11), 2862-2870
Abbaszadegan MR, et al. 2007 World J Gastroenterol., Vol 13(10), 1528-1533
Huang Z, et al. 2007 Dig Dis Sci., Vol 52(9), 2287-2291
Huang Z H, et al. 2007 World J Gastroenterol., Vol 13(6), 950-954
Itzkowitz SH, et al. 2007 Clin Gastroenterol Hepatol., Vol 5(1), 111-117
Zou H, et al. 2007 Cancer Epidemiol Biomarkers Prey. 16: 2686-2696.
Kisiel J B, et al. 2012 Cancer. 118(10):2623-31.
Boynton K A, et al. 2003 Clin Chem. 49(7):1058-65.
Han, Y. W., et al. 2000. Immun. 68:3140-3146.
Bolstad, A., et al. 1996. Clin. Microbiol. Rev. 9: 55-71.
Kostic A D, et al. 2012 Genome Res. 22(2):292-8.
Castellarin M, et al. 2012 Genome Res. 2012 22(2):299-306.
McCoy A N, et al. 2013 PLoS One, 8(1) : e53653.

We claim:

1. A method for detecting the presence of colorectal neoplasm in a subject, said method comprising:
(a) Collecting stool sample from said subject; and
(b) Quantifying fusobacterium nucleatum (FNN) DNA in said stool sample; wherein the quantity of said FNN DNA in a given amount of said stool sample must be above a quantitative level (threshold) to indicate the presence of said colorectal neoplasm in said subject.

2. The method of claim 1, wherein the said colorectal neoplasm is an adenomatous lesion or polyp.

3. The method of claim 1, wherein the said colorectal neoplasm is colorectal carcinoma.

4. The method of claim 1, wherein said subject is a human.

5. The method of claim 1, wherein said quantitative level (threshold) is experimentally determined.

6. The method of claim 1, wherein said quantitative level (threshold) is more than 1 copy of FNN DNA detected.

7. The method of claim 1, wherein said quantitative level (threshold) is more than 10 copies of FNN DNA detected.

8. The method of claim 1, wherein said quantitative level (threshold) is more than 100 copies of FNN DNA detected.

9. The method of claim 1, wherein said quantitative level (threshold) is more than 300 copies of FNN DNA detected.

10. The method of claim 1, wherein said quantitative level (threshold) is more than 1000 copies of FNN DNA detected.

11. A method for detecting the presence of colorectal neoplasm in a subject, said method comprising:
(a) Collecting stool sample from said subject; and
(b) Quantifying fusobacterium nucleatum (FNN) DNA in said stool sample; wherein the quantity of said FNN DNA in a given amount of said stool sample must be above a quantitative level (threshold) to indicate the presence of said colorectal neoplasm in said subject; and
(c) Detecting the presence of one or more other biomarkers in said stool sample, wherein the presence of said one or more other biomarkers together with the quantification of said FNN DNA in said stool sample is indicative of said colorectal neoplasm in said subject.

12. The method of claim 11, wherein said colorectal neoplasm is an adenomatous lesion or polyp.

13. The method of claim 11, wherein said colorectal neoplasm is colorectal carcinoma.

14. The method of claim 11, wherein said subject is a human.

15. The method of claim 11, wherein said quantitative level (threshold) is experimentally determined.

16. The method of claim 11, wherein said one or more other biomarkers are selected from the group consisting of human DNA mutations, quantity of human DNA in a given amount of said stool sample, quantity of total DNA in a given amount of said stool sample, fecal occult blood markers, and human genes having aberrant methylation.

17. The method of claim 11, wherein said one or more other biomarkers are K-ras mutations, the quantity of human DNA in a given amount of said stool sample, the quantity of total DNA in a given amount of said stool sample, and fecal occult blood markers.

18. The materials for detecting the presence of colorectal neoplasm in human, said materials comprising:
(1) materials necessary for quantifying the quantity of fusobacterium nucleatum (FNN) DNA in a given amount of stool; and
(2) materials necessary for detecting and/or quantifying one or more biomarkers in said stool.

19. The method of claim 18, wherein said one or more other biomarkers are selected from the group consisting of human DNA mutations, quantity of human DNA in a given amount of said stool sample, quantity of total DNA in a given amount of said stool sample, fecal occult blood markers, and human genes having aberrant methylation.

20. The method of claim 18, wherein the gene used for quantifying the amount of said FNN DNA in a given amount of said stool is nusG gene (Genbank Accession Number AAL94126.1) of said FNN genome (ATCC 25586).

21. The method of claim 18, wherein said materials comprising primers
(1) 5′-CAACCATTACTTTAACTCTACCATGTTCA-3′
(2) 5′-ATTGACTTTACTGAGGGAGATTATGTAAAA-3′
which are necessary for quantitatively amplifying said nusG gene (Genbank Accession Number AAL94126.1) of said FNN genome (ATCC 25586) in a given amount of said stool.


SEQUENCE LISTING (6 sequences)

SEQ ID NO: 1 - DNA, 29 bp, Fusobacterium nucleatum
caaccattactttaactctaccatgttca

SEQ ID NO: 2 - DNA, 30 bp, Fusobacterium nucleatum
attgactttactgagggagattatgtaaaa

SEQ ID NO: 3 - DNA, 24 bp, Homo sapiens
ggtaggtttgtagccttcatcacg

SEQ ID NO: 4 - DNA, 24 bp, Homo sapiens
cttgagaggtagagtgtggtgttg

SEQ ID NO: 5 - DNA, 23 bp, Homo sapiens
atcgtcaaggcactcttgcctac

SEQ ID NO: 6 - DNA, 24 bp, Homo sapiens
gtactggtggagtatttgatagtg